(19) United States
(12) Patent Application Publication
(10) Pub. No.: US 2021/0375471 A1
(43) Pub. Date: Dec. 2, 2021

(54) SYSTEM AND METHOD FOR ASSESSING PATIENT HEALTH STATUS AND RELATING HEALTH RISK TO CARE SERVICES

(71) Applicant: SAS IOTIED, Cupertino, CA (US)

(72) Inventor: Rabi Chakraborty, Cupertino, CA (US)

(73) Assignee: SAS IOTIED, Cupertino, CA (US)

(21) Appl. No.: 17/335,672

(22) Filed: Jun. 1, 2021

Related U.S. Application Data
(60) Provisional application No. 63/031,894, filed on May 29, 2020.

(51) Int. Cl. G16H 50/30; G16H 40/20

(52) U.S. Cl. CPC G16H 50/30; G16H 40/20

(57) ABSTRACT

A system and method for assessing health risks and rapidly providing care services. In an example the method, health data is received from a plurality of medical sources, including medical devices, sensor, and patient records. At least one risk assessment tool is selected using a tool evaluator. The selected risk assessment tools are used to identify any medical risks. The identified medical risks are communicated to a bot. The bot is configured to identify events, messages and actions to perform based on the health risks. The bot generates a task agenda for addressing the health risks using patient health delivery services.

### BACKGROUND

The healthcare industry has continuously evolved in terms of the collection and use of digital patient data. Health data may be obtained on-site with a medical professional and assessed by the medical professional as soon as the data is available. Oftentimes, the medical professional does not have a full set of data with which to make an assessment as certain types of data may take time to process. For example, diagnostic tests may require having samples sent to a laboratory for analysis that may take several days to perform. Once analyzed, the patient may need to return to see the medical professional again to receive the assessment information and suggested options for care services.

Disease progression, however, is a biologically and continuously occurring event. The progression of disease conditions may occur between patient visits and such progression may require urgent care services. Additional data may also be available, but not collected while the patient waits to be informed of the health assessment.

Many disease-related events may be captured using various biomedical devices and sensors, which may provide medical data relating to the patient. Processes for using such—only recently available—data continue to evolve. There remains however, a considerable gap in time between an assessment of health data, a determination of medical risks, and provision of care services for patients in today's healthcare industry.

### BRIEF DESCRIPTION OF THE DRAWINGS

FIG. 1 is a block diagram of an example system for assessing and rapidly providing health care services.

FIG. 2 is a flow diagram illustrating operation of an example method for assessing and rapidly providing health care services.

FIG. 3 is a table illustrating evaluation of risk engine tools for identifying medical risk.

FIG. 4 is a table illustrating a risk engine output in an example implementation.

FIG. 5 is a block diagram of another system for assessing and rapidly providing health care services.

### DETAILED DESCRIPTION

Described herein are examples of systems and methods for providing health assessments and rapidly delivering care services to patients. Human health data may be received from a variety of sources, including for example wearable devices with sensors, diagnostic equipment being used by the patient, and a variety of databases that may store, for example, patient records and medical information. The health data may be analyzed using a software platform incorporating in an example implementation machine learning and artificial intelligence methods. The health data is analyzed to identify health risks. The health risks may then be correlated with probable proactive care services using a bot. The bot may engage in communication with humans, electronic devices and machines intelligently. A health care delivery interface may then make suggested care services available to eligible humans.

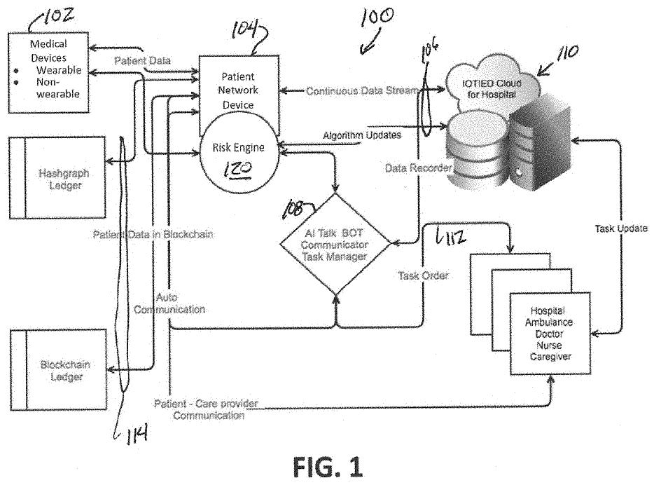

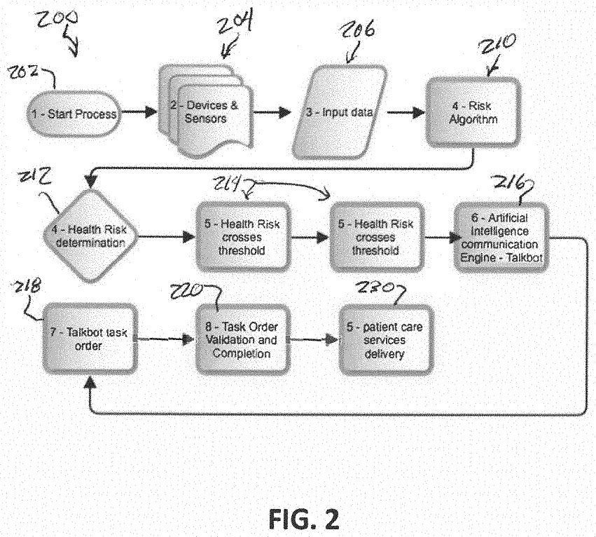

FIG. 1 is a schematic diagram of an example implementation of a system 100 for assessing medical risks and providing care service. FIG. 2 is a flow diagram 200 illustrating operation of an example process for assessing health data, assessing medical risks, and providing care service, which may be performed in an example implementation of the system in FIG. 1.

Referring to FIGS. 1 and 2, the system includes a plurality of medical devices 102, which may include wearable or non-wearable devices having sensors or elements that detect chemical, biological or physical modalities related to a patient's health status. The system also includes a patient network device 104 (e.g. smart phone or other, preferably portable, computerized device), a database interface 106, a communicator and task manager 108, a hospital data cloud 110, and a care delivery interface 112. The system may receive data input from the medical devices to determine the status of various health parameters of the patient on a real time, virtually continuous basis. For example, wearable devices may provide data such as heart rate, blood pressure, PO2 and other parameters substantially in real-time.

The system may also receive relevant medical data from, for example, patient records, and other databases as shown at 114. The patient records and other relevant information may be stored in ledger databases, relational databases, or other suitable databases.

The data may be provided to the user's patient network device, such as a smartphone, for pre-processing and compiling with other relevant medical data. The user's patient network device may operate, or communicate with, a risk engine 120 that performs a risk algorithm. The risk algorithm analyzes the received data from the patient and the databases to determine medical risks present to the patient. Example implementations of a risk algorithm are described below.

As shown in FIG. 2, the process starts at 202 with devices initiating connections via wifi or other networking infrastructure, with medical devices, which may be wearable devices, non-wearable devices, devices having sensors that contact the patient body, and some operating independently or stand-alone apart from the patient as shown at 204. An auto discovery procedure may be performed to discover devices and sensors to enable the data input from the devices and sensor at step 206. In an example implementation, a self-discovery procedure may use IPV6 standard protocols. Standard protocols such as IPV6 may allow the system to discover a sensor or a device, ping the device, and request a connection to the device. If permitted, the devices and sensors may complete a connection and begin to communicate.

The risk engine (FIG. 1) may operate on the patient network device, or on a separate networked component according to the risk algorithm at step 210. The risk engine receives the input health data and analyzes the health data to determine medical risks associated with the patient. Different health data and different medical conditions may be processed using different risk assessment tools. Some medical conditions and associated health data may best be analyzed using regression while others may best be suited for analysis using classification. In an example implementation, the risk engine determines assessment tools based on three factors:

1. Ease of interpretation of the output
2. Time to calculate results
3. Predictive power of the tool

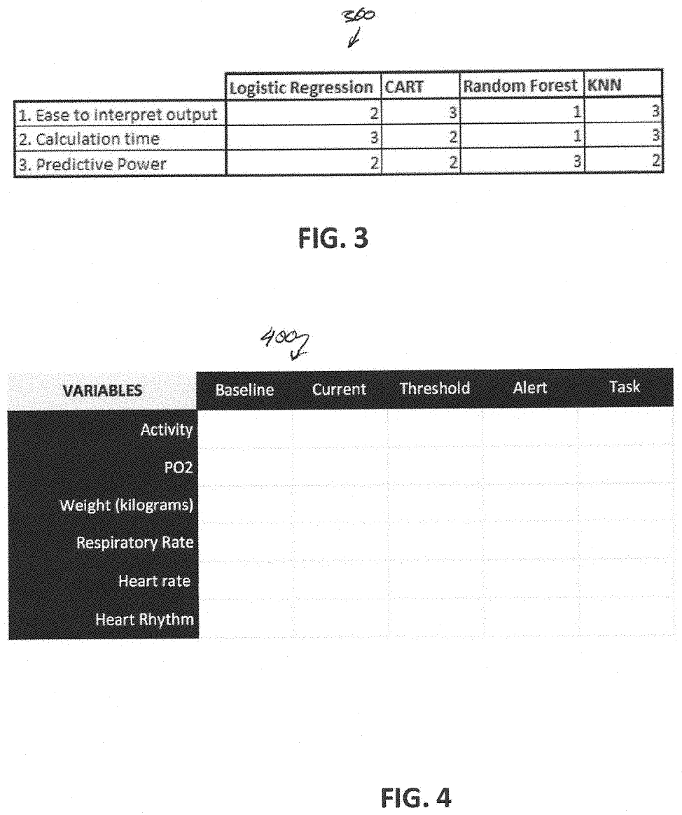

The risk engine may perform an evaluation of risk assessment tools and identify one or more risk assessment tools with which to provide various layers of health risks. The evaluation of the risk assessment tools may make a determination of the tools to use based on a table such as tool evaluation table 300 in FIG. 3 where the risk engine evaluates the use of either a Classification and Regression Tool (CART), a logistic regression tool, a random forest tool, or a Kernel normalization norm tool based on the above three factors. It is noted that the identified tools are listed as examples of the types of tools that can be used. The list of examples is not intended as limiting in scope. Others, or fewer tools, may also be evaluated and used in other implementations.

The risk engine (FIG. 1 and ref. no. 210 in FIG. 2—Risk Algorithm) uses one or more of the above-mentioned tools and the health data that is input to the system to generate layers of health risk at step 212 in a manner illustrated in FIG. 4. The model 400 illustrated in FIG. 4 is intuitive and dynamic. The model is dynamic in that it uses machine learning and artificial intelligence derived from the data sets collected over time. The parameters may be modified in the baseline and the threshold columns in FIG. 4. The current data column may then be loaded with current data corresponding to the rows in FIG. 4 to arrive at alerts and tasks that need to be performed. The model changes based on actions taken over time.

The layers of health risk illustrated in FIG. 4 may then be merged to provide risk scores to various conditions as shown in Table 1 below. Each relevant health condition (based on the patient data) may be provided a risk score relative to a high and low limit. These risk scores may change dynamically, virtually in real-time. The updated last scores will be used to define tasks and alerts to be assigned to an individual.

TABLE 1

| | Risk score | Risk score |
|---|---|---|
| Description | limit high | limit low |
| Cardiovascular superset risk | 90 | 65 |
| Congestive heart failure | 75 | 55 |

Once medical risks have been identified by the risk engine (FIG. 1), health risks that cross a threshold at step 214 in FIG. 2 may be identified (at no. 5 in FIG. 2). More than one medical risk may be identified. At step 216 in FIG. 2, a dialog may then be started with the bot to identify any care services that may be relevant. The bot may engage in communication with humans, electronic devices and machines intelligently, as if the machine is a human. A simulated version may use medically validated commands to communicate. The bot component may be implemented to function as a set of events, risk levels, messages, and actions. Table 1 below illustrates an example of the type of output that may be provided by the bot component.

TABLE 2

| | | Health | | |
|---|---|---|---|---|
| Description | Event | Risk level | Messaging | Action |
| Cardiovascular | Increased | Elevated to | Automated | Dispatch |
| condition | blood | 78% | machine phone | Ambulance |
| | pressure | | Call to | |
| | | | individual | |
| | Abnormal | Elevated | Automated | Dispatch |
| | ECG | blockage | machine phone | ambulance |
| | | of artery | call to individual | and arrange |
| | | | Send text | emergency |
| | | | message to | hospitali- |
| | | | hospital care | zation |
| | | | manager | |

Each row in Table 2 may include information derived by using a series of internal software processes. The steps to achieve the desired actions may be decided by level of health risks. Each health risk level may determine, for example if an ambulance needs to be dispatched to a patient or if a nurse needs to be deployed at the patients location. The change in health vital parameter readings (indicated by the Event column in Table 2, such as blood pressure, glucose level, ECG, Heart rate, Heart Rhythm, Breathing rate, blood oxygen saturation, etc., keeps changing in each human body over time. Such changes may occur without notice but are recorded using wearable devices and then the new data sets determine a new health status or condition.

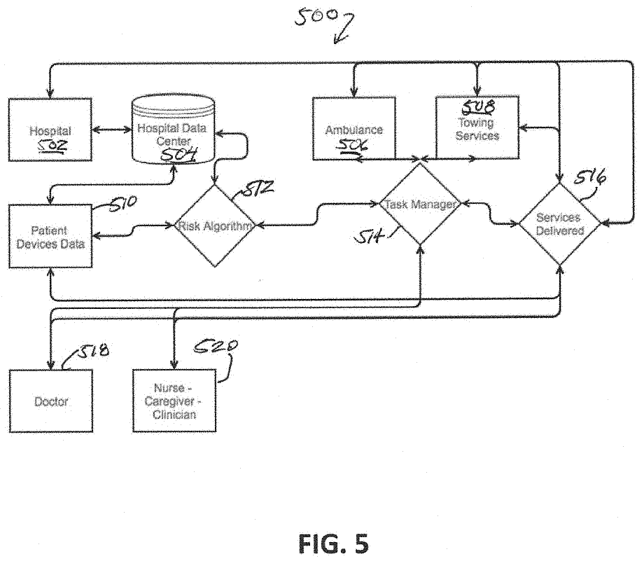

The bot may also provide a task list or series of tasks that may be performed to provide the patient with the appropriate care service. An infrastructure for providing care services is illustrated in FIG. 5. FIG. 5 depicts the risk algorithm (or engine) communicating with patient data (e.g. wearable devices, etc.), a hospital data center, and a task manager, which may be part of or integrated with the bot. The task manager communicates with services such as, an ambulance service, a doctor(s), nurses/caregiver, and a towing service. The task manager may also track services delivered.

As described above with reference to Table 2, the bot communicates with other agencies (ambulance services, doctors, nurses, etc.). The bot may also electronically create task orders to such agencies or parties to execute desired task orders at step 218 in FIG. 2. The tasks can be dynamically created and set up based upon a given subject. For example, an ambulance may be dispatched to attend to a critical patient at a given location identified from GPS location of an individual.

The tasks created by the task manager may be evaluated and monitored by the system at step 220 based on the medical risks identified by the risk engine (see Table 2). The set of actions in each line item may be aggregated into a task agenda, which is dependent upon health risk values calculated using mathematical algorithms. Such algorithms will change from time to time based upon data sets collected over time and actions suggested over time. The tasks in the task order list may then be communicated to patient care services to deliver the ordered services at step 230 in FIG. 2.

FIG. 5 is a block diagram of another system 500 for assessing and rapidly providing health care services. FIG. 5 illustrates a flow of communication of health information in the system 500 between example providers and services. As shown in FIG. 5, a hospital 502 may provide and access data from a hospital data center 504. The hospital data center 504 may communicate data with the patient devices data sources 510, which may include medical devices and sensor. The risk engine 512 incorporating examples of the risk algorithm and the bot described above may receive and communicate data with the hospital data center 504 and the patient devices data 510. The risk engine 512 may determine the health risks from the patient data and records as described above using the risk algorithm and the bot and create a task agenda. The task agenda may be communicated to a task manager 514. The task manager 514 may communicate with patient and health services providers to deliver services at 516. In an example implementation, a doctor 518 and nurses/caregivers/clinicians 520 may be provided access to the patient devices data, patient records, task agendas, and services ordered/delivered.

The disclosure provided herein describes features in terms of preferred and exemplary embodiments thereof. Numerous other embodiments, modifications and variations within the scope and spirit of the appended claims will occur to persons of ordinary skill in the art from a review of this disclosure.

What is claimed is:

1. A computer-implemented method for assessing health risks and rapidly providing care services, the method comprising:
receiving health data from a plurality of medical sources;
selecting at least one risk assessment tool using a tool evaluator;
using the risk assessment tool to identify any medical risks;
communicating the identified medical risks with a bot;
generating a task agenda based on the communications with the bot.

2. The method of claim 1 further comprising:
communicating orders to at least one agency based on an action in the task agenda.

3. The method of claim 2 further comprising:
monitoring performance of the actions performed by the at least one agency.

4. The method of claim 1 where in the step of selecting the at least one risk assessment tool, the tool evaluator includes a machine-learning algorithm to:
determine ease of interpretation of the output of the risk assessment tool;
determines a time to calculate results by the risk assessment tool; and
assess a predictive power of the risk assessment tool.

5. The method of claim 1 where the step of selecting the at least one risk assessment tool includes selecting a tool from one or more of a Classification and Regression Tool (CART), a logistic regression tool, a random forest tool, or a Kernel normalization norm tool based on the above three factors.

6. The method of claim 1 where the step of using the risk assessment tool further comprises:
generating layers of health risks based on the health data; and
merging the layers of health risks to provide risk scores to various health conditions.

7. The method of claim 6 where the step of using the risk assessment tool further comprises:
identifying any health risks that have crossed a threshold identified for the health risk.

8. The method of claim 7 where the step of communicating the health risks to the bot comprises communicating health risks that have crossed the threshold to the bot.

9. The method of claim 8 where the step of generating the task agenda comprises:
identifying an event corresponding to each communicated health risk;
determining a health risk level for the health risk;
establishing a communication to a corresponding health service for addressing the identified health risk; and
ordering an action corresponding to the health service.
